(19) United States
(12) Patent Application Publication
(10) Pub. No.: US 2004/0255344 A1
(43) Pub. Date: Dec. 16, 2004

(54) Rodent with urinary disturbance and method of constructing the same, and method of screening remedy for urinary disturbance using the rodent

(76) Inventors: Miyuki Sakai, Ibaraki (JP); Atsuhiko Kubota, Ibaraki (JP); Takahisa Hanada, Ibaraki (JP)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/491,640

(22) PCT Filed: Oct. 1, 2002
(86) PCT No.: PCT/JP02/10228

(30) Foreign Application Priority Data
Oct. 2, 2001 (JP) 2001-306485

(51) Int. Cl. A01K 67/027

(52) U.S. Cl. 800/14; 800/18

(57) ABSTRACT

It is intended to provide an animal model of urinary dysfunction induced by damage in brain nerve tissue in a small animal conveniently and at a high reproducibility. A rat in which the central urinary function alone is specifically damaged by regionally injuring the brain superior colliculus with the use of a legion generator. The animal model of urinary dysfunction is useful in a method of screening a therapeutic agent for urinary dysfunction.

### TECHNICAL FIELD

The present invention relates to a rodent with urinary dysfunction, and a method of producing the same, and more particularly to the rodent with urinary dysfunction induced by damaging a particular site of a brain, and a method of producing the same.

### BACKGROUND ART

The muscles that form the bladder may be mainly classified into the urinary muscles that primarily form the main part of the bladder (bladder smooth muscles), and the urethral sphincter that primarily forms the urethra. As a result of complex neural functions that control these muscles, accumulation of urine in the bladder and excretion occur in a voluntary or involuntary manner. Urinary dysfunction is a condition in which these functions do not act smoothly. Onset of this condition may have a variety of reasons including physiological aging, urological diseases, and neural dysfunctions, such as neural degenerative disease. The presence of urinary dysfunction has physically and psychologically deleterious effects on the patient, and lowers the quality of life (QOL). Moreover, because the aging of society is accelerating and there will only be an increasing number of patients suffering from urinary dysfunction, there is an increasing need for a therapeutic agent for urinary dysfunction.

A variety of urinary dysfunction models have been utilized thus far as systems to clarify physiological and pharmacological aspects of urinary reflex and to assess the therapeutic agent for urinary dysfunction. Models to directly stimulate bladder smooth muscle or the peripheral nervous system include, for example, chemical stimulant methods (acetic acid, capsaicin, and cholinergic reagents, etc.), electrical stimulation methods (electric stimulation of regulatory nervous system, etc.), and surgical stimulation methods (urethral stenosis model, etc.).

Models that provide disturbance of the central nervous system of the animal have been disclosed, including for example a method that inject a neurotoxin (ibotenic acid) (For example, refer to Non-Patent Literature 1), a decerebrate model (For example, refer to Non-Patent Literature 2), and a destructive method based on insertion of an electrode in a brain of a cat and then applying electricity (For example, refer to Non-Patent Literature 3). Hardly any of these urinary dysfunction models based on disturbance of the central nervous system are available in terms of the high death rates of the model animals and of the simplicity of the experimental techniques involved. Moreover, these models often provide complications with the damage of other brain functions from the point of the view of the urinary nervous system because it is extremely difficult to limit the region of brain injury for technical reasons.

For this reason, there is currently no satisfactory animal model which makes it possible to assess compounds with pharmacologically sufficient reliability, in which those are effective for the treatment of urinary dysfunction, and there is a earnest demand for the establishment of a highly reliable animal model for pharmacological evaluation.

In this regard, recently a lesion generator has been used to make an experimental brain infarction model in Mongolian gerbils (For example, refer to Non-Patent Literature 4). Nonetheless, the object described in this disclosure was to create an ischemia model by inducing an infarction in the brain, and nothing at all is described about introducing a specific injury of central nervous system by producing organic damage of the brain.

With the foregoing in view, the present invention focuses on small experimental animals such as a rodent that is easily handled as a pathological model, and it is an object of the present invention to provide an easy and highly reproducible urinary dysfunction model in conjunction with central nervous system injury. It is also another object to provide a method to produce the animal model.

Moreover, it is a further object of the present invention to provide a method of screening a therapeutic agent for urinary dysfunction using the urinary dysfunction animal model.

Non-Patent Literature 1: Hara et al., J. Pharmacol. Suppl., 1, 58, 1992

Non-Patent Literature 2: Yoshiyama et al., Eur. J. Pharmacol., vol 264, p467, 1994

Non-Patent Literature 3: Tang et al., J. Comparative Neurology, 106, 213-245, 1956

Non-Patent Literature 4: Iyoda et al., Cerebral Apoplexy, vol. 2(2), p93-95, 1980

### DISCLSOURE OF INVENTION

The above object is achieved by a rodent in which the neural tissues of a superior colliculus region of a brain has been damaged, inducing urinary dysfunction.

According to a preferable aspect of the present invention, aforementioned damage to the rodent is produced by a lesion generator.

According to a preferable aspect of the present invention, the damage to the rodents is conducted by applying electricity through an electrode of a lesion generator.

According to a preferable aspect of the present invention, the rodent is selected from the group consisting of rat, mouse and guinea pig.

Moreover, the above object is achieved with a rodent that has urinary dysfunction, the rodent produced by a method including the steps of (1) anesthetizing the rodent, (2) inserting a lesion generation electrode in the neural tissue of a superior colliculus region of a brain of the rodent under anesthetization, and (3) applying electricity through the electrode so that the tissue is damaged.

According to a preferable aspect of the present invention, the step of applying electricity in the method of producing the rodent is conducted under the conditions of an electrode temperature of 65 to 80° C. and an application time of 2 to 4 minutes.

According to a preferable aspect of the present invention, prior to the insertion step in the method of producing the rodent, a forming step is included in which an opening for passing through the electrode is formed by drilling into the cranium above the superior colliculus region.

According to a preferable aspect of the present invention, the rodent is selected from the group consisting of rat, mouse and guinea pig.

Moreover, the above object is achieved by a method to produce a rodent having urinary dysfunction which comprises the steps of (1) anesthetizing the rodent, (2) inserting a lesion generation electrode in the neural tissue of a superior colliculus region of a brain of the rodent under anesthetization, and (3) applying electricity through the electrode so that the tissue is damaged.

According to a preferable aspect of the present invention, the step of applying electricity in the production method is conducted under the conditions of an electrode temperature of 65 to 80° C., and an application time of 2 to 4 minutes.

According to a preferable aspect of the present invention, prior to the insertion step in the production method, a forming step is included in which an opening for passing through the electrode is formed by drilling into the cranium above the superior colliculus region.

According to a preferable aspect of the present invention, the rodent in the production method is selected from the group consisting of rat, mouse and guinea pig.

Moreover, the above object is achieved by a method of screening a therapeutic agent for urinary dysfunction, which comprises the steps of (1) administering a test substance into a body of a rodent having impaired urinary function, and (2) measuring the extent of recovery of urinary function after a constant time has consumed.

According to a preferable aspect of the present invention, damage is made in the neural tissue of the superior colliculus region of the brain of the rodent in the screening method in order to induce urinary dysfunction.

According to a preferable aspect of the present invention, the damage of the rodent in the screening method is conducted by the step of applying electricity under the conditions of an electrode temperature of 65 to 80° C., and an electrification time of 2 to 4 minutes.

According to a preferable aspect of the present invention, the measuring step of the screening method is conducted by measuring the volumes of the bladder.

According to a preferable aspect of the present invention, the rodent of the screening method is selected from the group consisting of rat, mouse and guinea pig.

The term “urinary dysfunction” used in the present invention is not given to disturbances of the smooth muscle of the bladder or to the amount of urine production of the kidney, but rather is based on functional disturbance of the urinary reflex between the bladder and the urinary central nervous system, and is a general way of referring to a rodent wherein acceleration of the urinary reflex has been induced and the remaining volumes of the bladder have no effect.

### BRIEF DESCRIPTION OF DRAWINGS

FIG. 1 indicates a front cross-sectional schematic diagram of a rat used in the present invention;

FIG. 2 is a diagram of the steps of the method of producing a rat having urinary dysfunction according to the present invention;

FIG. 3 is a schematic diagram of a system to measure the status of urinary reflex used in the present invention;

FIG. 4 is an expanded pattern diagram of the results of a cystometrogram measured by the system indicated in FIG. 3;

FIG. 5 is a diagram comparing typical cystometrograms of the normal urinary reflex state and an accelerated urinary reflex state. The arrows in FIG. 5 indicate the beginning of excretion;

FIG. 6 is a diagram indicating the results of inducing damage by a lesion generator to the neural tissue of the superior colliculus region of a rat brain according to the present invention;

FIG. 7 is a diagram indicating a description and the extent of urinary dysfunction induced after local destruction of the superior colliculus region of a rat brain according to the present invention;

FIG. 8 is a diagram indicating the results of the reproducibility of a urinary dysfunction model rat in which the neural tissue of the superior colliculus region of the rat brain has been damaged by the production method according to the present invention; and

FIG. 9 is a diagram indicating the results of the bladder volume of rat five minutes after intravenously injecting oxybutynin hydrochloride using a urinary dysfunction model rat according to the present invention.

### BEST MODE FOR CARRYING OUT THE INVENTION

Preferable embodiments of the present invention will be explained in detail below. The animals having urinary dysfunction targeted by the present invention are rodents, but in order to simplify the description, a rat will be used in the explanation below.

The rat used in the present invention is not particularly limited in sex or age, but from the perspective of obtaining a reproducible urinary dysfunction model, a rat weighing 150 to 300 g is preferable, and more preferable is 200 to 250 g.

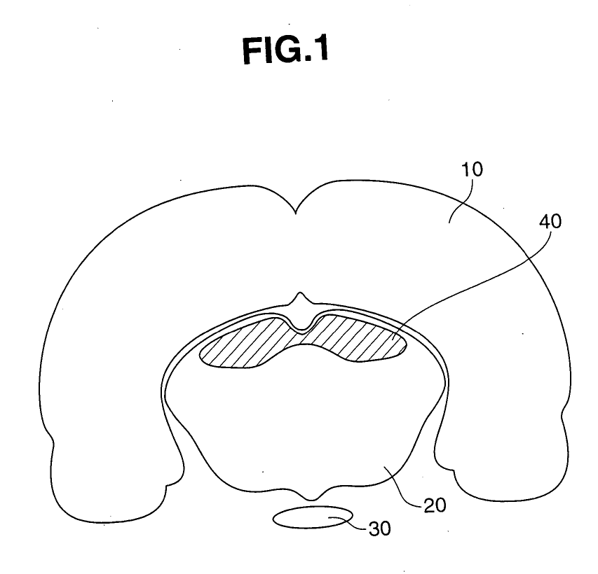

FIG. 1 indicates a schematic diagram of a rat brain map used in the present invention. The region of the brain is specified by the coordinates of the brain map, and it is possible to insert therein micro-electrode of a lesion generator to be described later, and it is possible to apply electricity to and destroy the superior colliculus region of the rat. As shown in FIG. 1, reference numeral 10 indicates the cerebrum of the rat; reference numeral 20 indicates the midbrain of the rat; and reference numeral 30 indicates the medulla oblongata. Reference numeral 40 of FIG. 1 corresponds to the superior colliculus region of the rat, and this is the region that is damaged by applying electricity using the lesion generator to be described later.

In the present invention, a pathological model with urinary dysfunction that quickly induces thamuria was discovered by using a lesion generator and applying electricity to the superior colliculus region 40 of the rat brain under specified conditions and causing localized damage to the superior colliculus region.

Here, we will explain the lesion generator used in the present invention. A lesion generator is a device that was made for the purpose of thermal coagulation of the test material by applying high frequency electric current of approximately 500 Hz from a TM type electrode connected to the main unit. The lesion generator is configured so that the temperature of the damaged tissue can be confirmed by a thermal sensor provided at the tip of the electrode. For this reason, it is possible to accurately control the damaging conditions. The lesion generator can impart damage of variable size to selectively targeted brain tissue.

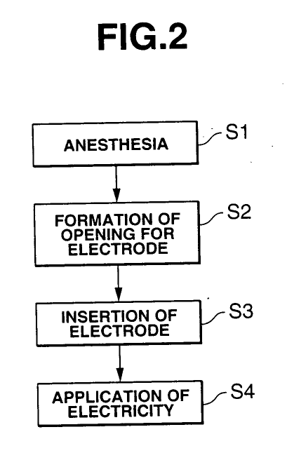

FIG. 2 indicates a diagram of the steps of the method of producing a rat having urinary dysfunction according to the present invention. As shown in FIG. 2, the method of producing the pathological model with urinary dysfunction first involves anesthetizing the rat with halothane, etc. (Step S1), and holding the head of the rat under anesthesia using a brain orientation-securing device.

Referring to the previously described rat brain map, a dental drill is used to form an opening in the cranium corresponding to the region above the superior colliculus region of the rat brain, through which the lesion generator electrode is passed (Step S2 of FIG. 2). At this time, the diameter of the superior colliculus region to be damaged is related to the size of the electrode of the lesion generator, and from the perspective of inducing highly reproducible urinary dysfunction based on the damage to be described later, 0.5 to 3.5 mm is preferable, and 0.8 to 3.0 mm is more preferable. The electrode of the lesion generator is not particularly limited, but a diameter of approximately 0.7 mm, and a length of approximately 1.5 mm are preferable.

Next, as shown in Step S3 of FIG. 2, the electrode is inserted into the superior colliculus region of the brain of the rat, the step of applying electricity is executed for a fixed time at a fixed current (Step S4 of FIG. 2), the neural tissue of the superior colliculus region of the rat brain is damaged, and urinary dysfunction is introduced into central nervous system. According to the present invention, it is possible to vary the conditions for damaging the neural tissue of the superior colliculus region of the brain of the rat, such as the electrode temperature and electrification time of the lesion generator, in order to produce a rat having highly reproducible urinary dysfunction.

The status of accelerating urinary dysfunction produced by the previously described urinary central nervous system damage can be confirmed by cystometrography after awakening from anesthesia.

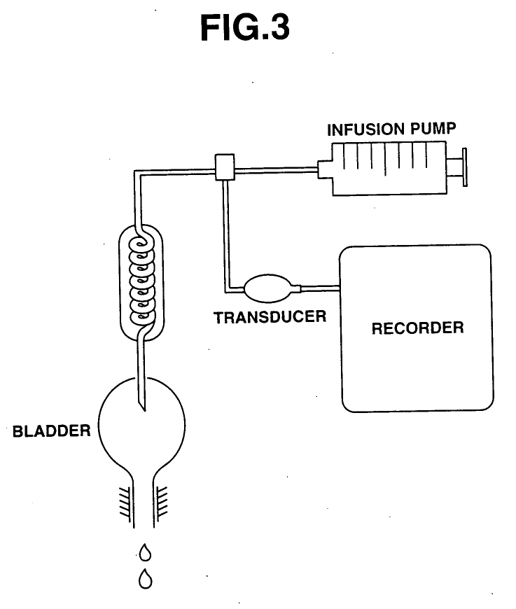

FIG. 3 indicates a schematic diagram of a system to measure the status of accelerated urinary reflex used in the present invention. As shown in FIG. 3, physiological saline is infuse into the bladder to be measured at a constant rate using an infusion pump, and changes of internal bladder pressure are measured through a transducer.

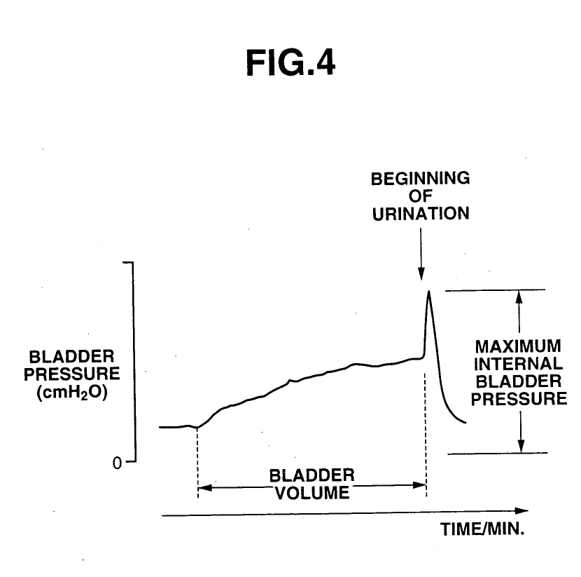

FIG. 4 is an expanded pattern diagram of the results of a cystometrogram measured by the system indicated in FIG. 3. Here, a cystometrogram is made when detecting the urinary reflex by putting a catheter (not indicated in the diagram) in the bladder, and continuously measuring the changes of internal bladder pressure through the said catheter.

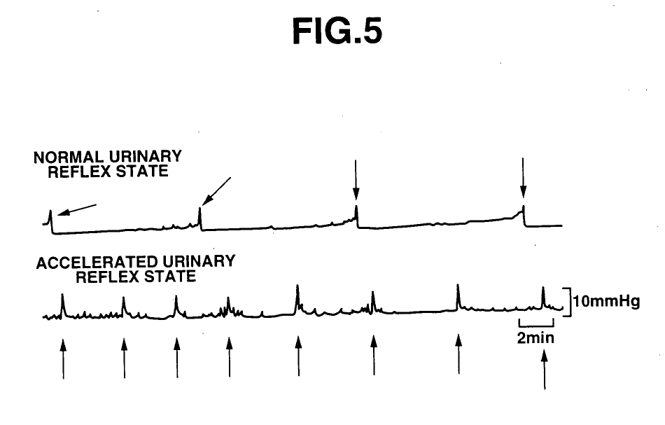

FIG. 5 indicates a diagram comparing typical cystometrograms of the normal urinary reflex state and an accelerated urinary reflex state. As demonstrated in FIG. 5, during the state of accelerated urinary reflex, the beginning of urination is frequently observed.

Further, the volume of the bladder can be calculated from the results of this cystometrogram as follows. Specifically, when infusing physiological saline into the bladder at a constant rate, the volume of the bladder can be calculated by multiplying the time required to reach the threshold where the urinary reflex starts by the fixed rate.

From the perspective of providing stable urinary dysfunction, the step of applying electricity according to the present invention has a preferable time of from 1.5 to 5 minutes, and more preferably, 2 to 4 minutes. Moreover, from the perspective of providing stable urinary dysfunction, the temperature in the step of applying electricity is preferably 55 to 85° C., and more preferably 65 to 80° C. The related temperature of the electrodes can be controlled corresponding to the current value from the lesion generator.

A rat having urinary dysfunction created by the present invention can be used to determine the effects of the reagents with a capacity to improve urinary dysfunction. Specifically, it is possible to screen a therapeutic agent for urinary dysfunction using rat based on the present invention.

The therapeutic agent to be tested is administered orally, intraperitoneally, subcutaneously or intravenously to a group of rats of the present invention and to a group of rats that are not based on the present invention and that are growing normally (called “control rat” hereinafter). It is possible to screen the therapeutic agent for urinary dysfunction by comparing the bladder volumes of the control rat and the rat of the present invention after a constant time has consumed.

The present invention will be explained more concretely below using examples, but the present invention is not at all limited by these examples.

### EXAMPLES

An urinary dysfunction model rat was produced by the following method. The rat (male, weight 220 g) was anesthetized using halothane, secured in a brain orientation-securing device, and median incision was made in the scalp. Then, following the coordinates of the rat brain map indicated in FIG. 1, a dental drill was used to make an opening (diameter approximately 1.0 mm) in the cranium corresponding to the site above the superior colliculus region. After a micro-electrode (diameter: 0.7 mm; length: 1.5 mm) of a lesion generator (Radionix model RFG-4) was inserted through the opening up to the superior colliculus region, then the neural tissue of the superior colliculus region of the brain of the rat was damaged by applying electricity at a variety of electrode temperatures for a variety of times.

After the operation was complete and the animals had awakened from the anesthesia, the post-operative rat was measured using cystometrography based on the system indicated in FIG. 3, and the status of accelerated urinary reflex was confirmed.

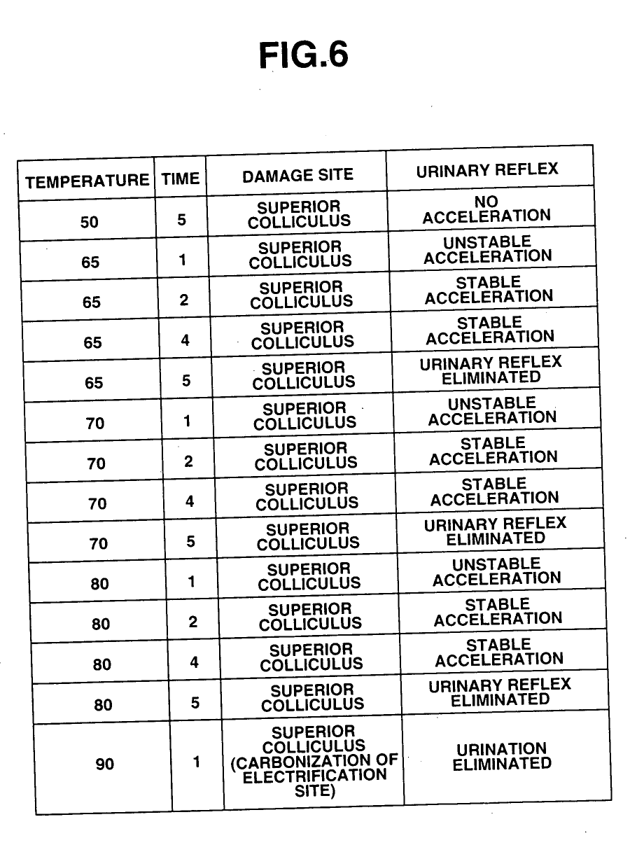

FIG. 6 indicates the results of the urinary dysfunction of rat subjected to surgery of the various conditions. The results of FIG. 6 demonstrated that adequate conditions for producing a urinary dysfunction model rat were when the electrode temperature of the lesion generator was 65° C. and the electricity application time was 4 minutes, thus producing a rat with more stable urinary dysfunction.

Further, “adequate conditions” used in the explanation below means the conditions when producing at a lesion generator electrode temperature of 65° C. and an electricity application time of 4 minutes.

After imparting localized destruction of the superior colliculus region of the brain of the rat using the adequate conditions obtained, the volume and extent of the introduced urinary dysfunction were observed and studied based on changes in the bladder volume, remaining amount of urine, and amount of urine produced (calculated from the sum total of the amount of remaining urine and the amount urine excreted).

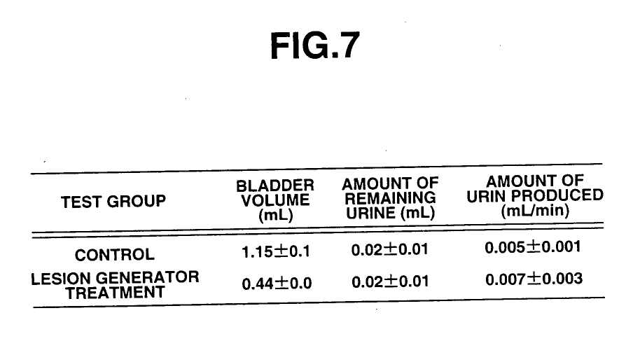

FIG. 7 indicates the results of comparing a urinary dysfunction rat produced under the previously described adequate conditions and a male control rat weighing 220 g undergoing normal growth. FIG. 7 demonstrates that the bladder volume of the rat produced by the present invention was smaller than that of the control rat, and thamuria was introduced. The results of FIG. 4 demonstrated that localized destruction of the superior colliculus region had no effect of the amount of remaining urine and the amount of urine produced, and that only the volume of the bladder was reduced. It may be understood that localized destruction of the urinary central nervous system function based on damage to the superior colliculus region accelerates the urinary reflex, and as a result reduces the bladder volume and induces thamuria.

Consequently, this confirms that the urinary dysfunction model rat produced by the present invention is a pathological model with urinary dysfunction that is caused by injury of the regulatory nervous system (This is similar to the urinary dysfunction shown in humans after neurological disease.), and is not caused by dysfunction of the motive organs such as the muscles associated with the urinary function.

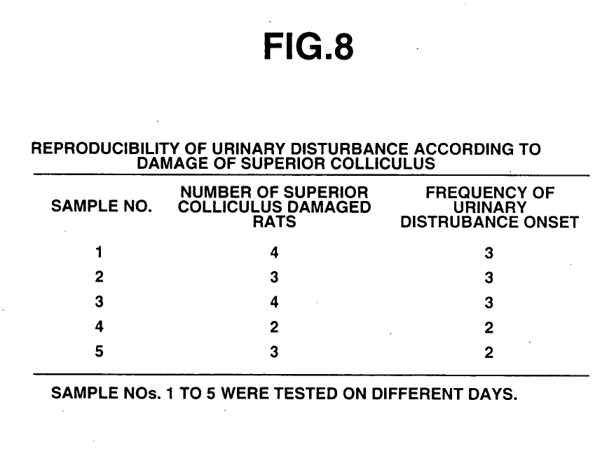

FIG. 8 is a diagram indicating the results of the reproducibility of a rat produced by the previous described adequate conditions. FIG. 8 demonstrated that the production method based on the present invention brings about urinary dysfunction in rat, and is highly reproducible.

Explained next are screening tests of a therapeutic agent for urinary disturbance using urinary dysfunction model rat produced as previously described.

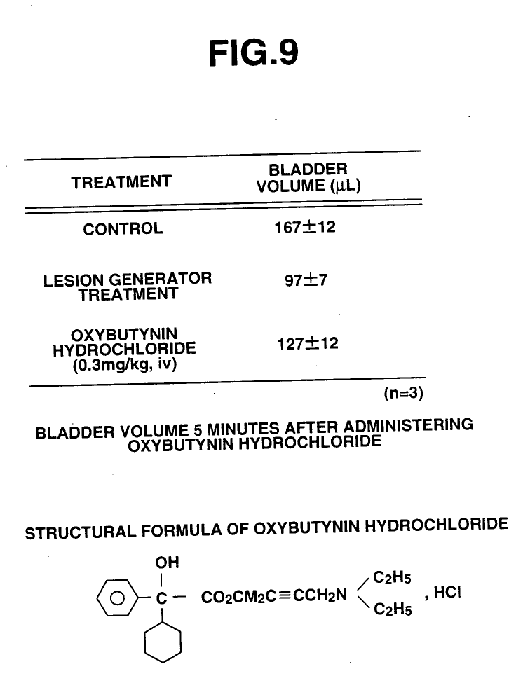

Using a control rat and the rat produced under adequate conditions based on the present invention, the presence of a therapeutic effect was assessed after intravenously administering oxybutynin hydrochloride (0.3 mg/kg iv:iv means intravenous injection, and iv means the same in FIG. 9.), which is a candidate compound as a therapeutic agent for urinary dysfunction.

FIG. 9 indicates the results of measuring the bladder volume of rat 5 minutes after intravenously administering oxybutynin hydrochloride. The results of administering oxybutynin hydrochloride to urinary dysfunction model rat based on the present invention demonstrated that the bladder volume of the rat in question increased, that the level of the urinary dysfunction was improved, and that oxybutynin hydrochloride is a compound with good prospects as the therapeutic agent for urinary dysfunction.

In this way, it is possible to conduct screening of the therapeutic agent for urinary dysfunction using urinary dysfunction model rat according to the present invention.

The above explanation used the rat, but a person skilled in the art can understand that it is possible to use rodents other than the rat.

### INDUSRTRIAL APPLICABILITY

From the above explanation, according to the production method based on the present invention, it is possible to offer a pathological model with urinary dysfunction based on damaging the neurological function of the brain, and it is possible to utilize that animal to screen compounds that are effective in treating urinary dysfunction.

We claim:

1. A rodent, comprising a neural tissue of a superior colliculus region of a brain being damaged in order to induce urinary dysfunction.

2. The rodent according to claim 1, wherein the damage is conducted by applying electricity through an electrode of a lesion generator.

3. The rodent according to claim 2, wherein the application of electricity is conducted under the conditions that an electrode temperature of the lesion generator is from 65 to 80° C., and an application time is from 2 to 4 minutes.

4. The rodent according to claim 1, wherein the rodent is selected from the group consisting of rat, mouse and guinea pig.

5. A rodent having urinary dysfunction produced by a method comprising the steps of:
(1) anesthetizing a rodent;
(2) inserting an electrode of a lesion generator into a neural tissue of a superior colliculus of a brain of the rodent under the anesthetization; and
(3) applying electricity through the electrode in order to damage the tissue.

6. The rodent according to claim 5, wherein the step of applying electricity is conducted under the conditions that an electrode temperature is from 65 to 80° C. and an application time is from 2 to 4 minutes.

7. The rodent according to claim 5, further comprising forming an opening for passing through the electrode by drilling into the cranium above the region of the superior colliculus region before the inserting step.

8. The rodent according to claim 5, wherein the rodent is selected from the group consisting of rat, mouse and guinea pig.

9. A method of producing a rodent having urinary dysfunction comprising the steps of:
(1) anesthetizing a rodent;
(2) inserting an electrode of a lesion generator into a neural tissue of the superior colliculus of a brain of the rodent under anesthetization; and
(3) applying electricity through electrode in order to damage the tissue.

10. The method according to claim 9, wherein the step of applying electricity is conducted under the conditions that an electrode temperature is from 65 to 80° C. and an application time is from 2 to 4 minutes.

11. The method according to claim 9, wherein prior to said inserting step, a step is included wherein a drill is used to make an opening in the cranium above the region of the superior colliculus through which the electrodes are passed.

12. The method according to claim 9, wherein the rodent is selected from the group consisting of rat, mouse and guinea pig.

13. A method of screening a therapeutic agent for urinary dysfunction comprising the steps of:
(1) administering a test substance into a body of a rodent having impaired urinary function, and
(2) measuring the extent of recovery of urinary function after a constant time has consumed.

14. The method according to claim 13, wherein a neuronal cell of a superior colliculus region of a brain of the rodent have been damaged in order to cause urinary dysfunction.

15. The method according to claim 14, wherein the damage is conducted by the step of applying electricity under the conditions that a temperature of a lesion generator electrode is from 65 to 80° C. and an electrification time is from 2 to 4 minutes, using a lesion generator.

16. The method according to claim 13, wherein measuring step of the screening method is conducted by measuring volumes of the bladder.

17. The method according to claim 13, wherein the rodent is selected from the group consisting of rat, mouse and guinea pig.
